(19) United States
(12) Patent Application Publication
(10) Pub. No.: US 2022/0287847 A1
(43) Pub. Date: Sep. 15, 2022

(54) DISC PROSTHESIS FOR CONTROLLED FUSION

(71) Applicant: Kamran Aflatoon, Corona del Mar, CA (US)

(72) Inventor: Kamran Aflatoon, Corona del Mar, CA (US)

(21) Appl. No.: 17/695,296

(22) Filed: Mar. 15, 2022

Related U.S. Application Data
(60) Provisional application No. 63/161,067, filed on Mar. 15, 2021.

(51) Int. Cl. A61F 2/44

(52) U.S. Cl. CPC A61F 2/4425; A61F 2002/443; A61F 2002/30892

(57) ABSTRACT

A lumbar and/or cervical disc prosthetic formed with three primary layers, including a superior (upper) plate, inferior (lower) plate, and intermediate core, in a sandwiched configuration. The superior plate member is adapted to be secured on one side to an upper vertebra in a spinal column, the inferior lower plate member is adapted to be secured on one side to a lower vertebra in the spinal column, and the core permits a limited degree of articulation of the superior plate relative to the inferior plate. Both the superior plate member and inferior plate member are constructed of two different materials, metal and plastic, such that the outward surfaces expose islands of metal surrounded by plastic to deter bone ongrowth to the plastic while promoting bone ongrowth to the metal islands. The prosthetic disc preserves spinal articulation years after the procedure.

### CROSS-REFERENCE TO RELATED APPLICATION(S)

The present application derive priority from U.S. provisional application Ser. No. 63/161,067 filed 15 Mar. 2021.

### BACKGROUND OF THE INVENTION

### 1. Field of the Invention

The present invention relates to artificial intervertebral disc implants and, in particular, to a prosthetic disc incorporating superior and/or inferior plate members that constrain bone ongrowth to certain predetermined regions to improve spinal articulation.

### 2. Description of the Background

Deterioration of an intervertebral disc can result from disease, trauma or aging, and symptoms include limited mobility, and severe pain. A herniated disc occurs when the outer sack partially ruptures and the interior of the sack expands, pushing part of the disc into the spinal canal. This condition is also known as a slipped disc, an intervertebral disc hernia, a herniated intervertebral disc, and a herniated nucleus pulposus. Intervertebral disc degeneration and vertebral trauma are common, and can often lead to other conditions such as tumors, necrosis, and endocrine conditions. The most common surgical options include discectomy, fusion, or a combination of the two. However, these surgical options are highly invasive and require prolonged hospitalization and recovery. More recently, various implants or prosthetics to replace or augment intervertebral discs have been proposed for replacing a part or all of a removed disc. This technique spares natural motion.

Intervertebral disc prostheses are oblate spherical structures that function as cushions between opposing spinal vertebrae. Some use a ball and socket arrangement. For example, U.S. patent application Ser. No. 10/997,823 for “ARTICULATING SPINAL DISC PROSTHETIC” by the inventor herein et al. shows a disc prosthetic including a superior (upper) plate, inferior (lower) plate, and intermediate layer, in a sandwich. The superior plate member is adapted to be secured on one side to an upper vertebra in a spinal column, and the inferior plate member is adapted to be secured on one side to a lower vertebra in the spinal column. Both plates have substantially flat sides exposed upwardly with one or mom protruding tines, posts or teeth for affixation to the respective vertebra. However, both the superior plate member and inferior plate member are invariably formed of metal (typically titanium). The intention is to recreate the natural motion of the patient's spine. Metallic prostheses have a high affinity for bone overgrowth. The present inventor has discovered over the course of years upper vertebra fuses across the entire superior plate member, and the lower vertebra fuses across the entire inferior plate member. This restricts natural motion. Many plastics such as polyetheretherketone (PEEK) do not directly bond to bone. What is needed is a disc prosthesis incorporating a superior plate member and inferior plate member constructed of two different materials, one the promotes bone ongrowth and one that deters it so as to selectively constrain bone ongrowth to predetermined subsections of the two plates. The underlying mechanical construct needed to accomplish the foregoing must facilitate manufacturing and yet provide a suitably robust prosthetic fit for a lifetime of use.

### SUMMARY OF THE INVENTION

Accordingly, it is an object of the present invention to provide a prosthetic disc incorporating a superior plate member and inferior plate member that constrain bone ongrowth to predetermined regions, deter bone ongrowth outside said regions, and thereby preserve spinal articulation years after the procedure.

It is another object to provide the foregoing by a mechanical construct that makes use of a plastic body with integrally-joined metal components that cannot come apart, is highly durable, and resistant to fatigue.

In accordance with the foregoing object, the present invention comprises a lumbar and/or cervical disc prosthetic formed with three primary layers, including a superior (upper) plate, inferior (lower) plate, and intermediate core, in a sandwiched configuration. The superior plate member is adapted to be secured on one side to an upper vertebra in a spinal column, the inferior lower plate member is adapted to be secured on one side to a lower vertebra in the spinal column, and the core permits a limited degree of articulation of the superior plate relative to the inferior plate. Both the superior plate member and inferior plate member are constructed of two different materials, metal and plastic, such that the outward surfaces expose islands of metal surrounded by plastic to deter bone ongrowth to the plastic while promoting bone ongrowth to the metal islands. This constrains bone ongrowth to limited regions of the two plates and improves long-term post-surgical mobility. Embodiments are proposed that accomplish the foregoing with metal insets that seat flush within plastic plate members, are co-molded therein, and secured by inward-projecting anchors. The metal insets bear outward-projecting tines to promote bone ongrowth and/or for screw-securement to the respective vertebra. The particular configurations described herein constrain bone ongrowth to predetermined regions of the superior plate member and/or inferior plate member, deter bone ongrowth to other regions, and thereby preserve spinal articulation years after the procedure. Moreover, the mechanical construct comprises a generally-plastic main body with integrally-joined metal insets that cannot come apart. The result is highly durable, resistant to fatigue, and yet allows a limited degree of articulation over a 30-40 year lifetime.

### BRIEF DESCRIPTION OF THE DRAWINGS

Other objects, features, and advantages of the present invention will become more apparent from the following detailed description of the preferred embodiment and certain modifications thereof, in which:

FIG. 1 is a side perspective view of an intervertebral disc implant 2 according to an embodiment of the present invention.

FIG. 2 is a side cross-section of the disc implant 2 of FIG. 1.

FIG. 3 is a side perspective view of an alternative embodiment of an intervertebral disc implant 20.

### DETAILED DESCRIPTION OF THE PREFERRED EMBODIMENTS

The present invention comprises intervertebral disc implants incorporating a superior plate member adapted to be secured on one side to an upper vertebra in a spinal column, and inferior lower plate member adapted to be secured on one side to a lower vertebra in the spinal column. In addition, a core sandwiched between the plates permits a limited degree of articulation. Both the superior plate member and inferior plate member are constructed of two different materials: metal and plastic, such that the outward surfaces expose islands of metal surrounded by plastic. The metal inlays promote 5 bone ongrowth while the surrounding plastic deters bone ongrowth. This constrains bone ongrowth over time to limited regions of the two plates and improves long-term post-surgical mobility.

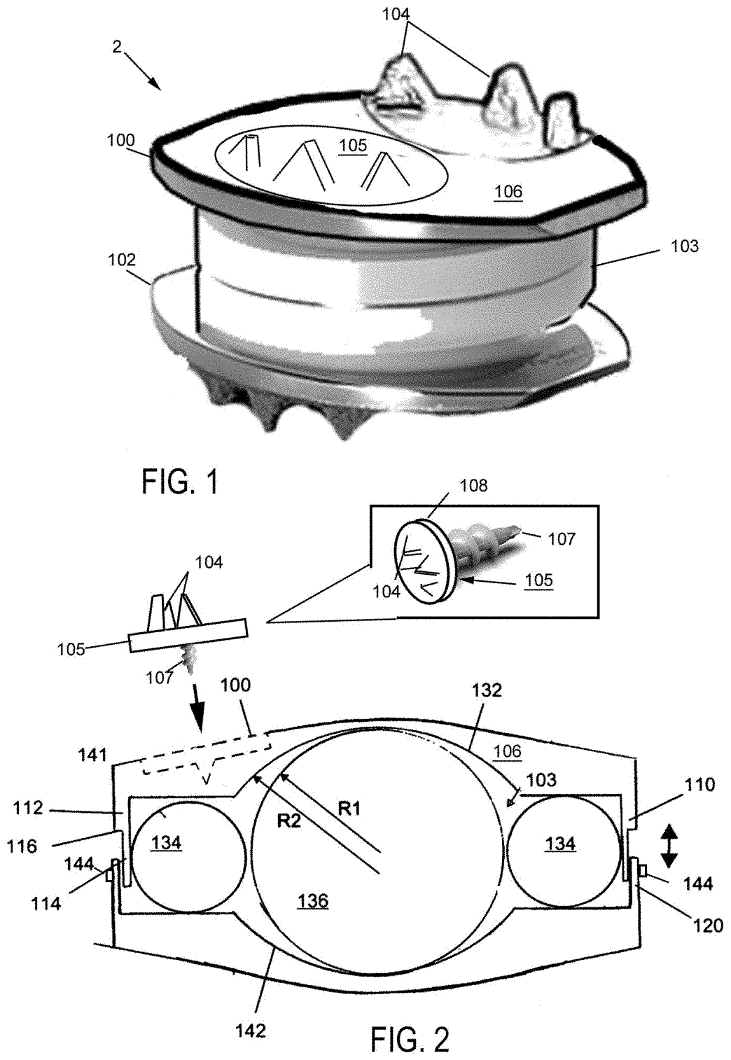

FIG. 1 is a perspective view of the intervertebral disc implant 2 according to a first embodiment of the present invention. FIG. 2 is a side cut-away view of the disc implant 2 of FIG. 1. With combined reference to FIGS. 1-2, a first embodiment of the present invention incorporates an upper, or superior, plate member 100, and a lower, or inferior, plate member 102, which are adapted to be secured to upper and lower vertebra, respectively, in a spinal column. An articulating member 103 is also provided, disposed between the upper and lower plate members 100, 102.

Each of upper and lower plate members 100, 102 are provided with a pattern-array of projections, tines or teeth 104 for securement to the upper and lower vertebra (not shown). Many types of securement means are known in the art, and could be used with the present invention. Known means of securing the plate members 100, 102 to the respective vertebra include the use of screws through the projections 104 and into the vertebral body, bone fusion of the vertebral body onto the projections, 104, or a combination of the two. In the illustrated embodiment the projections 104 are arrayed in triplet patterns on each side of each plate member 100, 102. Each projection 104 comprises a triangular tooth, and the teeth within each triplet are offset and rotated by about 10 degrees relative to each other as shown.

In accordance with the present invention either one or both of the superior plate member 100 and inferior plate member 102 are constructed of two different materials, metal and plastic, such that the outward surfaces expose islands of metal surrounded by 5 plastic to deter bone ongrowth to the plastic while promoting bone ongrowth to the metal islands. In the illustrated embodiment this is accomplished by molding the main body 106 of each plate member 100, 102 of plastic, preferably polyetheretherketone (PEEK) plastic which does not directly bond to bone, and co-molding metal insets 105 into the plastic main body 106. As an alternative to PEEK plastic, the main body 106 may be made of ceramics (e.g., hydroxy apatite), other polymers, or any other suitable material that does not promote bone ongrowth.

The metal insets 105 are preferably formed of titanium and include a round flat head 108 from which a triplet of triangular teeth 104 project upward, and a downward-projecting anchor 107 for maintaining engagement with the main body 106. The projecting anchor 107 may be a screw-anchor or any other anchor with lateral flanges to secure it within the main body 106. The metal insets 105 are co-molded so that the round flat head 108 is recessed within the plastic main body 106 such that the outward-facing surfaces of the superior plate member 100 and inferior plate member 102 are smooth and continuous except for projecting teeth 104. This way, the outward surfaces of main body 106 expose islands of metal surrounded by plastic which deters bone ongrowth to the plastic main body 106 yet promotes bone ongrowth onto the teeth 104 and head 108 of metal insets 105. This constrains bone ongrowth to the insets 105 and improves long-term post-surgical mobility. One skilled in the art will readily understand that insets 105 need not be co-molded with main body 106 but may be screw-inserted therein, provided that main body 106 is formed with a recess to seat the flat head 108 of inserts 105 and the anchor 107 is screw-threaded. The foregoing mechanical construct combines a generally-plastic main body 106 with integrally-joined metal insets 105 that cannot come apart. The result is highly durable, resistant to fatigue, and yet allows relative articulation. Toward this end upper and lower plate members 100, 102 may be respectively provided with interfitted (telescoping) sidewalls 110, 120 extending inward (toward one another) from the opposing surfaces of the plate members to form a housing for seating and containing a balloon or resilient member 103. The sidewalls 110, 120 are of sufficient height to overlap one another when the balloon or resilient member 103 is fully expanded after implantation. One or both of the sidewalls 110, 120 may be formed with a thicker portion 112 proximal to the plate from which is extends and a thinner distal end 114 for overlapping with the opposing sidewall. The transition from the proximal portion 112 to the distal portion 114 provides a positive stop 116 against which the distal end of the opposing sidewall my rest. The positive stop 116 provides a maximum limit that the intervertebral disc implant may articulate in a particular direction as a result of compression of the inner resilient member 103. The maximum limit provided by the positive stop 116 may be the same in all directions as depicted in the embodiment of FIGS. 1-2. It should be observed that while the depicted embodiment shows the lower sidewall extending outside and over the distal end of the upper sidewall the intervertebral disc implant 2 could be constructed such that the distal end of the upper sidewall extends outside and over the lower sidewall with similar efficacy.

Sidewalls 110, 120 may also be formed, as depicted in FIG. 2 with a radial sequence of protruding guides 144 (two are shown) formed in the outer surface of the distal end of sidewall 110. Guides 144 are captured within vertical slots in the sidewalls 120 of the opposing plate 102. The protruding guides 144 slide along the vertical slots and prevent relative rotation of the plates 100, 102, but permit a limited degree of pivoting flexion. Slots and guides 144 may be utilized prevent separation of the upper and lower plate members 100, 102 during articulation and control the limits thereof. Variation in the length of the slots is used to control the limits of articulation. In a preferred embodiment four slot and guide 144 pairs are implemented, one each at the cardinal points about the intervertebral disc implant 2 although more or less, including zero slots/guides may be used. Additionally, walls 110, 120 may be formed with a predetermined coefficient of friction on the overlapping portions in order to augment the resilient member 103 in controlling or restricting movement between the upper plate 100 and lower plate 102.

The upper plate member 100 preferably has a lower surface formed with a concave impression 132 that is complementary to the shape of the resilient member 103. The lower plate member 102 is likewise preferably formed with a lower surface having a concave impression 142 that is similarly complementary to the shape of the resilient member. In a preferred embodiment upper and lower plate members 100, 102 are circular in plan such that the space enclosed by the intervertebral disc implant 2 is substantially cylindrical although other forms are contemplated as described below. The space enclosed by intervertebral disc implant 2 is occupied by the resilient member 103. Resilient member 103 may be formed with a toroid encircling a sphere that fits within a central aperture of the toroid. The entire member 103 may be formed as an integral component or as two discrete components that are fitted together. The resilient member 103 may be made of any of a variety of known biocompatible resilient compounds such as silicone rubbers, polyether and polyester urethane, polymethyl methacrylate, polycarbonates and various other polymerizing resins or hydrogels having the desired elastic properties. Further, the individual members 134, 136 may each be selected from an elastomer having different elastic properties to achieve the desired operation of the intervertebral disc implant 2 as described blow.

In a preferred embodiment resilient member 103 is formed as a multi-chamber balloon having a toroidal chamber 134 encircling a spherical chamber 136. Both chambers 134, 136 are defined by expandable side walls made, for example, of silicone rubber. Both chambers 134, 136 may be inflated either before, or preferably, after implantation, chamber 134 inflating to establish the toroidal shape and chamber 136 inflating to create the spherical shape. In combination the inflated chambers of balloon 103 define an oblate spheroid shape which approximates the shape of the disc it is meant to replace. The chambers may be inflated with air (or another gas) or with any of a variety of liquid or viscous substances as well as curing resins to achieve the desired elastic properties as described. Further, the toroidal chamber 134 and the spherical chamber 136 may each be inflated with different substances to achieve the desired operation.

In use the resilient member is compressed within the intervertebral disc implant 2 by a retaining clip or similar so as to present a smaller overall height to ease insertion into the evacuated intervertebral space. After insertion the clip is removed to permit the resilient member to expand and the implant to return to its operative dimensions. Where the resilient member is a balloon, the implant may be inserted with the balloon deflated with the chambers being inflated after the device is positioned. Inflation may be accomplished by insertion of a syringe (not shown) through a port into each chamber so as to inject a fluid or resin filler.

The spherical member 136 when inflated seats itself within the opposing concave impressions 132, 142, in the upper plate member 100 and lower plate member 102, respectively. In a preferred embodiment impressions 132, 142 are formed with a slightly greater radius (R2) than the spherical member 136 to afford a limited degree of pivoting freedom for flexion. This way, the upper vertebrae may shift either laterally or in a front or rearward direction, relative to the lower vertebrae. This flexion is facilitated by the interfitted (telescoping) sidewalls 110, 120 extending inward from the major surface of the plates 100, 102. The telescoping sidewalls 110, 120 are free to slide together/apart as described.

Spherical chamber 136 acts a shock absorbing member with the shock absorbing ability a function of by the elastic properties of the chosen elastomer or balloon material and filler. As the spine is articulated, for example rotated forward in the sagittal plane during daily use, the shoulders 141, 144 of the upper and lower plate members 100, 102 formed about the periphery of the concave impressions 132, 142, engage the surface of the toroidal chamber 134. The elastic properties of the chosen elastomer or balloon material and filler of the toroidal chamber 134 determine the resistance of the implant 2 to this flexion. By choosing the relative and absolute elastic properties of the two resilient members the surgeon may customize the operational characteristics of the implant as both a shock absorber and an articulating joint to match the natural properties of patients original intervertebral disc and meet the needs of the patient.

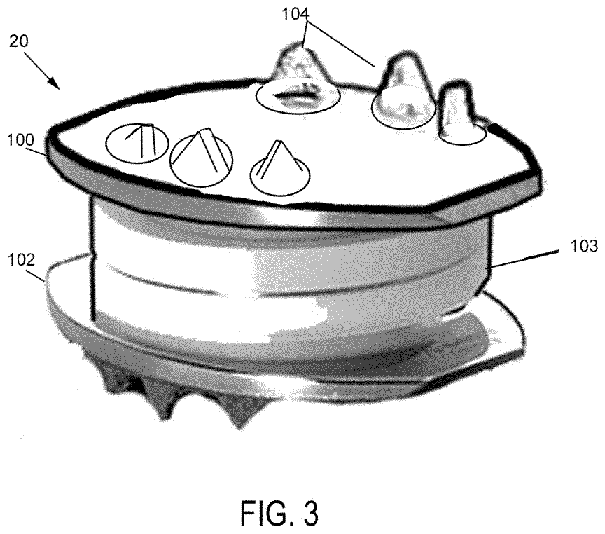

FIG. 3 is a perspective view of an alternative embodiment 20 in which multiple insets 105 are provided each having just one projection 104, the collective insets 105 still providing triplet patterns of projections 104 on each side of each plate member 100, 102. In all other respects the insets 105 are the same, but this configuration is more desirable where a smaller island size and less surface area for bone ongrowth is desired.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

I claim:

1. A multi-piece disc nucleus implant for replacing a nucleus removed by a discectomy, comprising:
an upper plate member formed primarily of a plastic main body with metal insets, the plastic main body having an outer surface and the metal insets seating flush within the plastic main body and secured therein by inward-projecting anchors, each said metal inset bearing one or more outward-projecting tines to promote bone ongrowth;
a lower plate member formed primarily of a plastic main body with metal insets, the plastic main body having an outer surface and the metal insets seating flush within the plastic main body and secured therein by inward-projecting anchors, each said metal inset bearing one or more outward-projecting tines to promote bone ongrowth; and
an intermediate core configured for providing unidirectional movement between the two plate members.
